(19) United States
(12) Patent Application Publication
(10) Pub. No.: US 2025/0258255 A1
(43) Pub. Date: Aug. 14, 2025

(54) Magnetic Susceptibility Measurement Device and Magnetic Susceptibility Measurement Method

(71) Applicant: TOHOKU UNIVERSITY, Sendai-shi (JP)

(72) Inventors: Shin Yabukami, Sendai-shi (JP); Kazuhiko Okita, Sendai-shi (JP); Toru Murayama, Sendai-shi (JP)

(21) Appl. No.: 19/193,387

(22) Filed: Apr. 29, 2025

Related U.S. Application Data
(63) Continuation of application No. PCT/JP2023/033188, filed on Sep. 12, 2023.

(30) Foreign Application Priority Data
Oct. 31, 2022 (JP) 2022-174443

(51) Int. Cl. G01R 33/16; G01N 27/72; G01R 33/12

(52) U.S. Cl. CPC G01R 33/16; G01N 27/72; G01R 33/1276

(57) ABSTRACT

A device for measuring magnetic susceptibility of an object. The device includes a probe including a signal transmission line formed therein with the object being disposed in proximity to or in contact with the signal transmission line and a magnetization easy axis direction of the object being orthogonal to the signal transmission line, a first unit for applying a magnetic field in the magnetization easy axis direction of the object, a second unit for applying a magnetic field in a magnetization hard axis direction orthogonal to the magnetization easy axis direction of the object, a signal measurement device for measuring a signal transmitting through the signal transmission line in a state in which magnetic fields are applied by both the first unit and the second unit, and an arithmetic processing means for obtaining the magnetic susceptibility of the object based on the signal measured by the signal measurement device.

### CROSS-REFERENCE TO RELATED APPLICATIONS

This application is a bypass continuation of International Patent Application No. PCT/JP2023/033188 filed Sep. 12, 2023, and claims priority to Japanese Patent Application No. 2022-174443 filed Oct. 31, 2022, the disclosures of which are hereby incorporated by reference in their entireties.

### BACKGROUND OF THE INVENTION

### Field of the Invention

The present invention relates to a magnetic susceptibility measurement device and a magnetic susceptibility measurement method for measuring the magnetic susceptibility of an object to be measured containing aggregates of magnetic particles.

### Description of Related Art

The measurement of magnetic susceptibility of magnetic nanoparticles with respect to the radio frequency (RF) has been widely studied (e.g., B. K. Kunar, V. Veerakumar and K. Lingam, S. R. Mishra, Alka V. Kuanr, R. E. Camley, and Z. Celinski, IEEE Transactions on Magnetics, Vol. 45, No. 10, OCTOBER (2009); M. Jadav, S. P. Bhatnagar, IEEE Transactions on Magnetics, vol. 56, p. 1-8 (2020); and P. C. Fannin, I. Malaescu and C. N. Marin, The European Physical Journal E, vol. 27, p. 145 to 148 (2008)).

The magnetic nanoparticles are so-called nano-sized magnetic particles, and are expected to be applied to, for example, magnetic hyperthermia, which is attracting attention as a therapeutic method for cancer, and an immunological test for detecting biological substances such as a derived from a disease or a pathogenic bacterium.

The magnetic hyperthermia is a therapy that can selectively necrotizing or degenerating only a cancer tissue by utilizing the fact that the cancer tissue is more vulnerable to heat than a normal tissue. For this purpose, the magnetic particles such as the magnetic nanoparticles are injected into a living body, and the magnetic particles absorbed in the living body are locally heated by application of an alternating magnetic field from the outside to selectively kill only the cancer tissue. As a result, the cancer tissue is treated.

A. Shikano, L. Tonthat, and S. Yabukami, IEEJ Trans. El. Electron. Eng., 16, 807 to 809 (2021) is a study of the present inventors and discloses a constant temperature heating control method for precisely controlling an applied magnetic field so as to maintain a constant temperature at a target temperature without overshooting and without fluctuation with respect to temperature control for heating the magnetic nanoparticles injected into the living body by application of the alternating magnetic field in an animal experiment.

In addition, in the immunological test for detecting the biological substance such as the protein derived from the disease or the pathogenic bacterium, an antigen-antibody reaction in which an antigen as a substance to be detected and an antibody specifically bind to each other is used, the antibody is labeled with a substance called a marker, and a signal from the marker of the antibody bound to the antigen is detected to measure the amount of the antigen. In the immunological test, a magnetic immunological test for detecting the substance to be detected by a magnetic method is a method for detecting the antigen-antibody reaction by using the magnetic particles and a magnetic sensor, in which the magnetic particles (hereinafter referred to as magnetic markers) are added to the antibody to label the antibody, and a degree of binding to the antigen is detected by a magnetic signal from the magnetic markers by the magnetic sensor by using a difference in Brownian relaxation characteristics of the magnetic markers.

Each of JP 2018-194305 A and 2 is a method proposed by the inventors of the present application, and discloses a magnetic field measurement device that rotates a sample containing the magnetic markers (magnetic nanoparticles) by a rotation mechanism and detects the antigen as an object to be measured by using Brownian relaxation by switching a magnetic field for each rotation cycle.

In the magnetic method using the magnetic particles as exemplified above, by optimizing the magnetic characteristics of the magnetic particles in a state of being aggregated, higher effects such as improvement in heating efficiency in the magnetic hyperthermia and improvement in measurement sensitivity in the magnetic immunological test can be obtained. Recently, the present inventors have developed a method of measuring the magnetic susceptibility of the magnetic particles in order to control the magnetic characteristics, particularly the magnetic susceptibility, of the magnetic particles.

It is therefore an object of the present invention is to provide a magnetic susceptibility measurement device and a magnetic susceptibility measurement method that can measure magnetic susceptibility of an object to be measured containing aggregates of magnetic particles.

### SUMMARY OF THE INVENTION

In a magnetic susceptibility measurement device for measuring magnetic susceptibility of an object to be measured containing magnetic particles, the magnetic susceptibility measurement device of the present invention for achieving the above-described object includes a probe including a signal transmission line formed therein, the object to be measured being disposed in proximity to or in contact with the signal transmission line so that a magnetization easy axis direction of the object to be measured is orthogonal to the signal transmission line, a first magnetic field applying unit for applying a magnetic field in the magnetization easy axis direction of the object to be measured, a second magnetic field applying unit for applying a magnetic field in a magnetization hard axis direction orthogonal to the magnetization easy axis direction of the object to be measured, a signal measurement device for measuring a signal transmitting through the signal transmission line in a state in which magnetic fields are applied by both the first magnetic field applying unit and the second magnetic field applying unit, and an arithmetic processing means for obtaining the magnetic susceptibility of the object to be measured based on the signal measured by the signal measurement device.

In a magnetic susceptibility measurement method for measuring magnetic susceptibility of an object to be measured containing magnetic particles, the method includes a step of disposing the object to be measured in proximity to or in contact with a probe including a signal transmission line formed therein, the object to be measured being disposed in proximity to or in contact with the signal transmission line so that a magnetization easy axis direction of the object to be measured is orthogonal to the signal transmission line, a step of applying, by a first magnetic field applying means, a magnetic field in the magnetization easy axis direction of the object to be measured, a step of applying, by a second magnetic field applying means, a magnetic field in a magnetization hard axis direction orthogonal to the magnetization easy axis direction of the object to be measured, a step of measuring, by a signal measurement device, a signal transmitted through the signal transmission line in a state in which the magnetic fields are applied by both the first magnetic field applying means and the second magnetic field applying means, and a step of obtaining, by an arithmetic processing means, magnetic susceptibility of the object to be measured based on the signal measured by the signal measurement means.

The present invention provides a magnetic susceptibility control method for controlling magnetic susceptibility of an object to be measured containing magnetic particles, and the method includes a step of magnetizing, by a first magnetic field applying means, the object to be measured in an easy axis direction of the object to be measured, a step of applying, by a second magnetic field applying means, a magnetic field in a magnetization hard axis direction orthogonal to the magnetization easy axis direction of the object to be measured, and a step of varying, by a control means, the strength of the magnetic field applied by the second magnetic field applying means to control to change the magnetic susceptibility of the object to be measured.

According to the present invention, magnetic susceptibility of an object to be measured containing magnetic particles can be measured. Further, the magnetic susceptibility of the object to be measured containing the magnetic particles is made variable, and thus the magnetic susceptibility can be controlled.

### BRIEF DESCRIPTION OF THE DRAWINGS

FIG. 1 is a diagram illustrating a schematic configuration example of a magnetic susceptibility measurement device according to an embodiment of the present invention.

FIG. 2 is a diagram illustrating a schematic configuration example of the magnetic susceptibility measurement device according to the embodiment of the present invention.

FIG. 3 is a diagram illustrating a configuration example of a probe 10.

FIG. 4 is a diagram illustrating a state in which an object to be measured 1 is disposed on the probe 10.

FIG. 5 is a flowchart illustrating a procedure of a measurement method of magnetic susceptibility according to the embodiment of the present invention.

FIG. 6 includes diagrams each showing a measurement result example of magnetic susceptibility κ.

FIG. 7 is a diagram showing a measurement result example of the magnetic susceptibility κ.

FIG. 8 includes diagrams each showing a measurement result example obtained by applying a magnetic susceptibility measurement method according to the present embodiment to a magnetic immunological test.

FIG. 9 includes diagrams each showing another measurement result example obtained by applying the magnetic susceptibility measurement method according to the present embodiment to a magnetic immunological test.

FIG. 10 is a graph showing a relationship between the magnetic susceptibility κ and a ferromagnetic resonance frequency (FMR) in the magnetic susceptibility measurement shown in FIG. 9.

FIG. 11 is a diagram illustrating a schematic configuration of a measurement device for carrying out a measurement example in a reference example.

FIG. 12 includes diagrams each showing a measurement result example (part 1) of the measurement example in the reference example.

FIG. 13 includes diagrams each showing a measurement result example (part 2) of the measurement example in the reference example.

### DESCRIPTION OF THE INVENTION

Embodiments of the present invention will be described below with reference to the accompanying drawings. However, these embodiments do not limit the technical scope of the present invention.

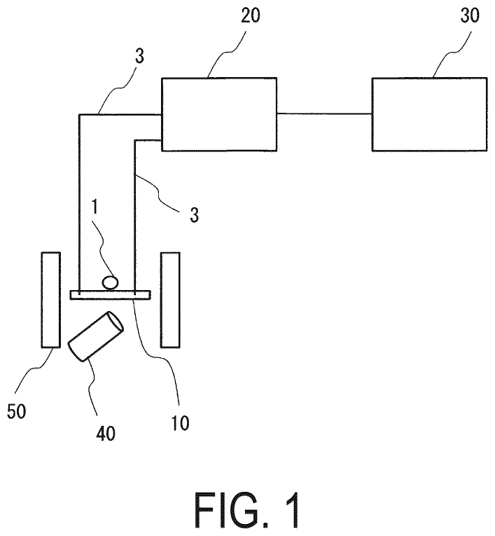

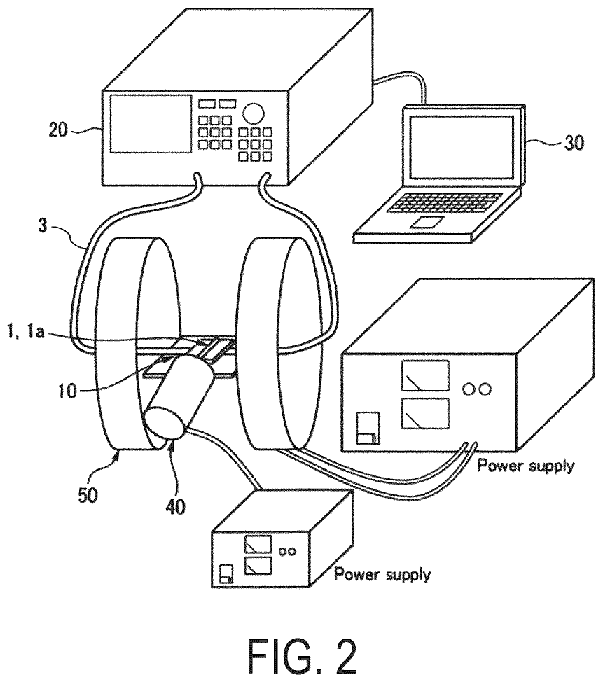

FIG. 1 and FIG. 2 are diagrams each illustrating a schematic configuration example of a magnetic susceptibility measurement device according to an embodiment of the present invention. FIG. 2 is a diagram more schematically illustrating a functional block diagram of FIG. 1.

The magnetic susceptibility measurement device according to the embodiment of the present invention includes a detecting probe 10 disposed in contact with or in proximity to an object to be measured 1, a network analyzer 20 as a signal measurement device, an arithmetic processing device (e.g., a computer device such as a personal computer) 30 for executing predetermined arithmetic processing such as numerical analysis processing, a magnetization easy axis coil 40 for applying a DC magnetic field in a magnetization easy axis direction of the object to be measured 1, and a magnetization hard axis coil 50 for applying a DC magnetic field in a magnetization hard axis direction orthogonal to the magnetization easy axis direction.

The probe 10 is disposed so as to be in contact with or in proximity to the object to be measured 1 containing aggregates of magnetic particles, and is connected to the network analyzer (for example, N5227A manufactured by Agilent Technologies Ltd.) 20 via a signal cable (for example, a coaxial cable) 3. A high-frequency current signal is supplied by the network analyzer 20 which is a current supply source to measure a transmission coefficient (S₂₁) of the object to be measured 1, the signal data thereof is taken into the arithmetic processing device (computer device) 30, and the magnetic susceptibility of the object to be measured 1 is obtained by predetermined numerical analysis processing. In addition, a magnet (magnetic field applying unit) 40 including a solenoid coil (electromagnetic coil) which is controlled to be energized for applying a magnetic field to the object to be measured 1 is used.

The arithmetic processing device 30 functions as an arithmetic processing means for obtaining the magnetic susceptibility of the object to be measured 1 and executes a predetermined arithmetic processing program for calculating the magnetic susceptibility. The arithmetic processing device 30 also functions as a control means for changing the intensity of the DC magnetic field applied by the magnetization hard axis coil 50 to control the magnetic susceptibility.

The magnetization easy axis coil 40 is an electromagnetic coil that supplies a DC magnetic field for magnetizing the magnetic particles contained in the object to be measured 1 disposed on the probe 10 in the magnetization easy axis direction.

The magnetization hard axis coil 50 is an electromagnetic coil that applies a DC magnetic field to the magnetic particles contained in the object to be measured 1 disposed on the probe 10 in the magnetization hard axis direction. FIG. 2 illustrates a power supply for the magnetization easy axis coil 40 and a power supply for the magnetization hard axis coil 50.

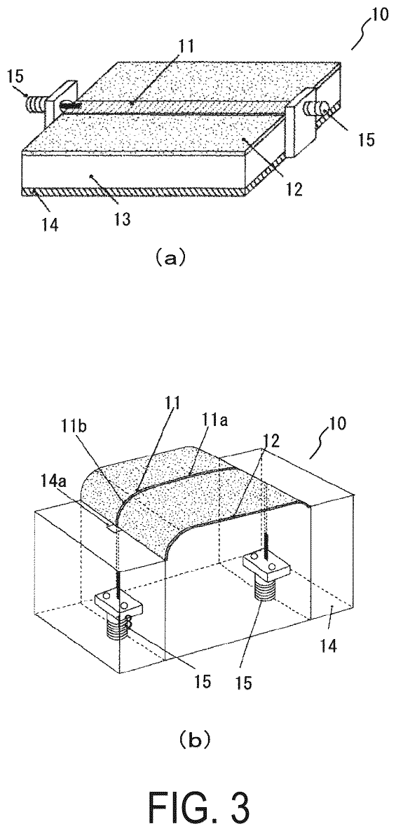

Each diagram in FIG. 3 illustrates a configuration example of the probe 10, FIG. 3(a) illustrates a form of the probe 10 of a first configuration example, and FIG. 3(b) illustrates a form of the probe 10 of a second configuration example. The first configuration example of the probe 10 illustrated in FIG. 3(a) includes a microstrip conductor 11, a flexible substrate 12, a fluororesin substrate 13, a ground conductor 14, and a pair of connectors 15 each connected to a respective one of both ends of the microstrip conductor 11. The microstrip conductor 11 is processed into a linear microstrip line by etching. The microstrip conductor 11 and the flexible substrate 12 are integrally fixed to each other by a chemical treatment or a thermal treatment. The ground conductor 14 is made of, for example, a copper foil. A configuration in which the flexible substrate 12 and the fluororesin substrate 13, which are dielectrics, are interposed between the microstrip conductor 11 and the ground conductor 14 forms a microstrip line.

The second configuration example of the probe 10 illustrated in FIG. 3(b) includes a microstrip conductor 11, a flexible substrate (sheet) 12, a ground conductor 14, and a pair of connectors 15 each connected to a respective one of both ends of the microstrip conductor 11. The connector 15 is connected to the signal cable 3 (FIGS. 1 and 2). The microstrip conductor 11 and the flexible substrate 12 are integrally fixed to each other by a chemical treatment or a thermal treatment. In the second configuration example, the fluororesin substrate 13 in the first configuration example of FIG. 3(a) is omitted, and the flexible substrate 12 is pressed against the ground conductor 14 having a planar structure and a curved surface structure. Although the inside of the ground conductor 14 is illustrated as transparent for the sake of explanation, it is actually made of a metal material such as copper. The microstrip conductor 11 is processed by etching. The microstrip conductor 11 includes a linear portion 11a at the center and curved portions 11b on both sides of the center. An end portion of the microstrip conductor 11 is electrically connected to the connector 15. Characteristic impedance of the microstrip conductor 11 is matched to 50Ω in both the linear portion 11a and the curved portions 11b. Similarly to the first configuration example, a configuration in which the flexible substrate 12, which is a dielectric, is interposed between the microstrip conductor 11 and the ground conductor 14 forms a microstrip line.

The microstrip conductor 11 extends into the ground conductor 14 through an opening 14a provided in the ground conductor 14 and is connected to the connector 15 on the opposite surface side. Regardless of the shape of the object to be measured 1, measurement can be performed without the object to be measured 1 colliding with the connector 15 and the signal cable 3 (FIG. 1) connected to the connector 15.

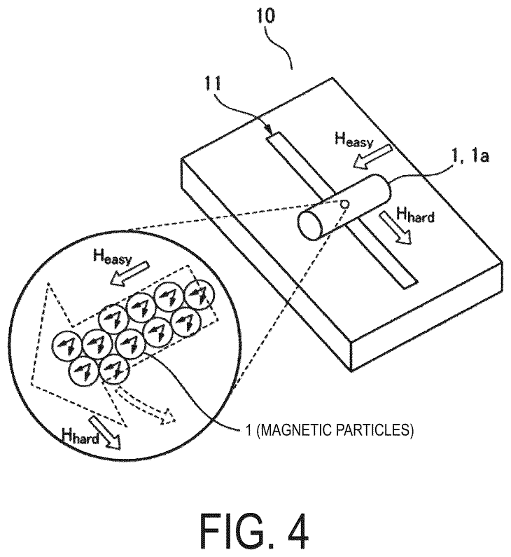

FIG. 4 is a diagram illustrating a state in which the object to be measured 1 is disposed on the probe 10. The object to be measured 1 is aggregates of magnetic particles enclosed in a microtube 1a (for example, about 5 UL in size and about 5 mm in length) which is an elongated cylindrical container. The magnetic particles are, for example, magnetic nanoparticles having a nano-sized particle (for example, 200 nmf), and are present in a state of being aggregated in a solution in the microtube 1a. For example, by bringing a permanent magnet in proximity to the microtube 1a, the magnetic particles in a state of being dispersed in the microtube 1 are attracted toward the permanent magnet, so that a state in which the magnetic particles are aggregated can be created.

As illustrated, the microtube 1a having the elongated cylindrical shape is oriented in a direction orthogonal to the direction in which the microstrip conductor 11 extends, and is disposed in proximity to or in contact with the microstrip conductor 11. The longitudinal direction of the microtube 1a is the magnetization easy axis direction of the magnetic particles contained therein, and thus by disposing the microtube 1a in the direction orthogonal to the microstrip conductor 11, the direction orthogonal to the microstrip conductor 11 is the magnetization easy axis direction, and the aggregates of the magnetic particles are magnetized in the magnetization easy axis direction by application of a magnetic field H_{easy} in the magnetization easy axis direction by the magnetization easy axis coil 40.

In a state in which the magnetic particles are magnetized in the magnetization easy axis direction, subsequently, a magnetic field H_{hard} in the magnetization hard axis direction orthogonal thereto is applied by the magnetization hard axis coil 50 to measure the magnetic susceptibility of the magnetic particles.

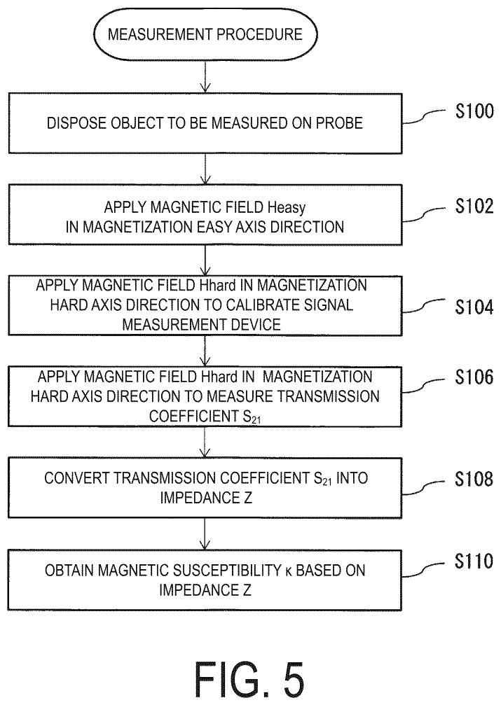

FIG. 5 is a flowchart illustrating a procedure of a measurement method of magnetic susceptibility according to the embodiment of the present invention.

The object to be measured 1 containing aggregates of the magnetic particles in the microtube 1a is disposed on the microstrip conductor 11 of the probe 10 (S100). As described above, the microtube 1a is disposed such that the longitudinal direction thereof is oriented in a direction orthogonal to the direction in which the microstrip conductor 11 extends, and the magnetization easy axis direction is orthogonal to the direction in which the microstrip conductor 11 extends.

The DC magnetic field H_{easy} is applied to the object to be measured 1 in the magnetization easy axis direction by the magnetization easy axis coil 40 to magnetize the magnetic particles of the object to be measured 1 in the magnetization easy axis direction (S102). The intensity of the DC magnetic field applied in the magnetization easy axis direction is, for example, 0.135 [T].

In a state in which the DC magnetic field H_{easy} is applied in the magnetization easy axis direction by the magnetization easy axis coil 40, subsequently the magnetic field H_{hard} is applied to the object to be measured 1 by the magnetization hard axis coil 50 in the magnetization hard axis direction in which the microstrip conductor 11 extends to calibrate the signal measurement device (network analyzer) 20 (S104). Specifically, a relatively strong DC magnetic field H_{hard} (for example, 0.4 [T]) is applied by the magnetization hard axis coil 50 and magnetically saturate the object to be measured 1 to calibrate the network analyzer 20. By doing so, the electrical lengths of the probe 10 and the coaxial cable 3, the direct current impedance of the object to be measured 1, the non-magnetic signal, and the like are removed. This calibration enables measurement based on a state in which a predetermined magnetic field is applied to the object to be measured.

The DC magnetic field H_{hard} is applied by the magnetization hard axis coil 50, and the transmission coefficient (S₂₁) of a high-frequency current signal in a state in which the magnetic field is applied by both the magnetization easy axis coil 40 and the magnetization hard axis coil 50 is measured (S106). Preferably, the transmission coefficient (S₂₁) in accordance with the frequency of the high-frequency current signal is measured by changing the strength of the magnetic field H_{hard} in the magnetization hard axis direction applied by the magnetization hard axis coil 50 in a state in which a constant DC magnetic field H_{easy} is applied in the magnetization easy axis direction by the magnetization easy axis coil 40. By the magnetic field H_{hard} applied by the magnetization hard axis coil 50, the transmission coefficient (S₂₁) can be measured, the transmission coefficient in accordance with the change in magnetic susceptibility according to a behavior in which the magnetization oriented in the magnetization easy axis direction rotates in the magnetization hard axis direction in a magnetic anisotropy. The magnetic susceptibility is obtained by arithmetic processing based on the measured transmission coefficient (S₂₁). In the arithmetic processing for obtaining the magnetic susceptibility, first, the transmission coefficient (S₂₁) is converted into impedance Z of the object to be measured 1 by the following equation (1) (S108).

Z=100(1−S₂₁)/S₂₁ (1)

To be more specific, in S104, the transmission coefficient (S₂₁) measured by applying a strong magnetic field by the magnetization hard axis coil 50 to saturate the object to be measured 1 is used as a background, and the transmission coefficient (S₂₁) at this time is used as a reference signal. Next, the intensity of the magnetic field applied by the magnetization hard axis coil 50 is changed to measure the transmission coefficient (S₂₁). The transmission coefficient (S₂₁) at this time reflects the magnetic characteristics by the magnetic field applied to the object to be measured 1, and the impedance Z reflecting the rotation of the magnetization of the magnetic particles in the object to be measured 1 can be obtained by the intensities of the magnetic field H_{easy} applied in the magnetization easy axis direction and the magnetic field H_{hard} applied in the magnetization hard axis direction orthogonal to the magnetization easy axis direction. Further, magnetic susceptibility κ is obtained based on the obtained impedance Z (S110).

In the impedance Z, its real part is a loss (resistance component) R of the object to be measured 1, and its imaginary part is a product ωL of an inductance component L and an angular frequency ω of the object to be measured 1. The inductance component L corresponds to the real part (κ′) of the magnetic susceptibility κ of the object to be measured 1, and the resistance component R corresponds to the imaginary part (κ″) of the magnetic susceptibility κ of the object to be measured 1. The magnetic susceptibility (complex number) K of the object to be measured 1 is expressed by the following equation (2).

κ=κ′−jκ″ (2)

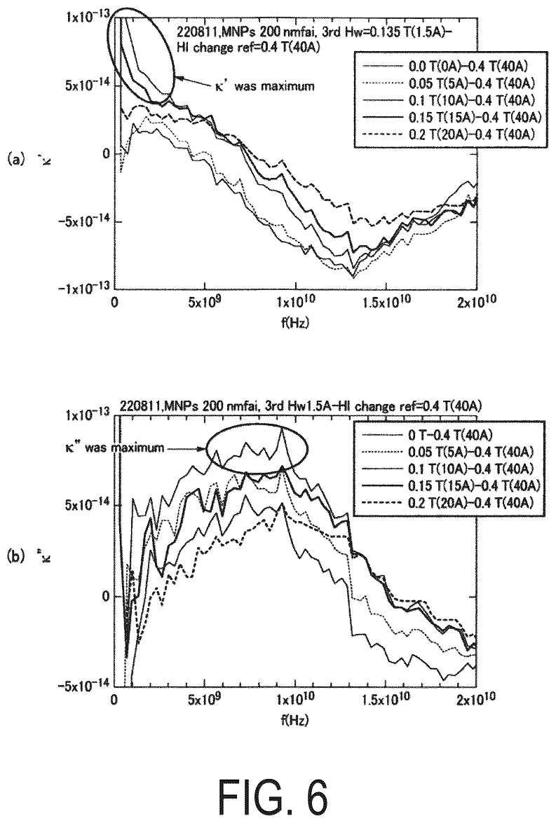

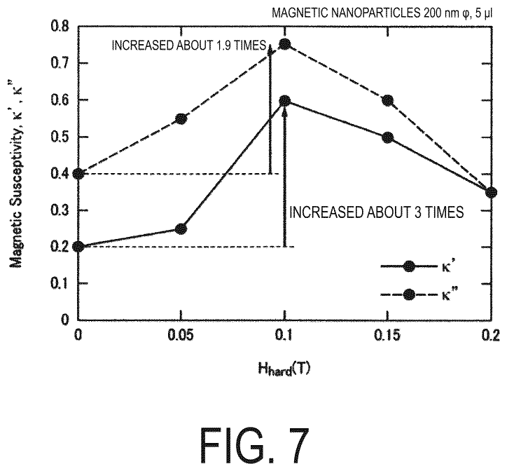

FIG. 6 and FIG. 7 are diagrams each showing a measurement result example of the obtained magnetic susceptibility κ. FIG. 6 shows frequency-dependence of the magnetic susceptibility κ when the strength of the magnetic field applied in the magnetization hard axis direction is varied. FIG. 6(a) shows a value of the real part (κ′) of the magnetic susceptibility κ. FIG. 6(b) shows a value of the imaginary part (κ″) of the magnetic susceptibility κ. FIG. 7 shows the magnetic susceptibility κ with respect to the intensity of the magnetic field applied in the magnetization hard axis direction in the magnetic susceptibility κ FIG. 6.

In the measurement result examples shown in FIGS. 6 and 7, a relative value (a.u.) of the magnetic susceptibility κ obtained by dividing the impedance Z by a frequency f is simply used as the value of the magnetic susceptibility κ on the vertical axis. In addition, as for an absolute value of the magnetic susceptibility κ, the magnetic susceptibility κ can be obtained by performing known electromagnetic field analysis processing (for example, finite element method analysis) and obtaining table data of a relationship between the magnetic susceptibility and the inductance in advance. In the table data of the magnetic susceptibility and the inductance, an inductance L when the magnetic susceptibility κ is changed can be obtained by calculation using Maxwell's equations by finite element method analysis processing. The computer device 30 obtains the table data showing the relationship between the magnetic susceptibility and the inductance by calculation and stores it as data.

FIG. 6 shows the magnetic susceptibility κ when the strength of the magnetic field applied in the magnetization hard axis direction is varied (including a case in which no magnetic field is applied in the magnetization hard axis direction (0.0 T)), and specifically shows the magnetic susceptibility κ when the strength of the magnetic field (magnetic flux density) in the magnetization hard axis direction is 0.0 T (no magnetic field applied), 0.1 T, 0.05 T, 0.1 T, 0.15 T and 0.2 T. In this measurement result example, the magnetic susceptibility κ also changes in accordance with the strength of the applied magnetic field, and in particular, when the strength of the magnetic field was approximately 0.1 T and 0.15 T, the magnetic susceptibility κ tended to increase, and in particular, the magnetic susceptibility κ became maximum at 0.1 T. From the fact that the intensity of the magnetic field applied in the magnetization easy axis direction is 0.135 T, it is considered that the case in which the intensities of the magnetic fields applied in the magnetization easy axis direction and the magnetization hard axis direction orthogonal to each other are substantially equal to each other corresponds to an anisotropic magnetic field, and the magnetic susceptibility κ is substantially maximized.

FIG. 7 is a graph plotting the maximum values of the magnetic susceptibility κ shown in FIG. 6, in which the real part (κ′) and the imaginary part (κ″) of the magnetic susceptibility κ are both increased by applying the magnetic field in the magnetization hard axis direction as compared with the case in which no magnetic field is applied in the magnetization hard axis direction. In addition, the magnetic susceptibility κ is also changed by changing the strength of the magnetic field in the magnetization hard axis direction, which suggests that the magnetic susceptibility κ can be controlled by changing the strength of the magnetic field.

By increasing the magnetic susceptibility of the object to be measured 1 which is the magnetic particles, for example, when the real part (κ′) of the magnetic susceptibility κ increases, a signal strength of the magnetic field of the object to be measured 1 increases, and an effect of improving detection sensitivity is obtained. For example, in the magnetic immunological test, it contributes to improvement in detection sensitivity of a signal from magnetic markers (magnetic particles) of an antibody bound to an antigen. In addition, for example, when the imaginary part (κ″) of the magnetic susceptibility κ increases, the loss (resistance) of the object to be measured 1 increases and the loss is converted into heat, thus contributing to, for example, an improvement in heating efficiency of the magnetic particles in the magnetic hyperthermia.

As described above, the magnetic susceptibility of the magnetic particles can be measured by the method of applying the magnetic field to the magnetic particles in the two axis directions of the magnetization easy axis direction and the magnetization hard axis direction, and the magnetic susceptibility of the magnetic particles that changes in accordance with the strength of the magnetic field in the magnetization hard axis direction can be measured by changing the magnetic field applied in the magnetization hard axis direction. Further, in the application of the magnetic field in the two axis directions of the magnetization easy axis direction and the magnetization hard axis direction, by changing the strength of the magnetic field in the magnetization hard axis direction, the magnetic anisotropy of the magnetic particles is controlled and the magnetic susceptibility of the magnetic particles is made variable, and thus particularly the magnetic susceptibility can be controlled so as to increase.

Specifically, in a case in which the magnetic particles are applied to the magnetic hyperthermia or the magnetic immunological test, a method for controlling the magnetic susceptibility of the magnetic particles (in particular, aggregates of the magnetic particles) first magnetizes the magnetic particles to be controlled in the magnetization easy axis direction, then applies the magnetic field in the magnetization hard axis direction, and varies the strength of the magnetic field in the magnetization hard axis direction at that time to change the magnetic susceptibility of the magnetic particles. This method improves the magnetic characteristics of the magnetic particles and provides useful and advantageous technical advances in the field of application of the magnetic particles, such as improvement in the heating efficiency of the magnetic particles in the magnetic hyperthermia, improvement in the detection sensitivity of the magnetic particles as the magnetic markers in the magnetic immunological test, and the like.

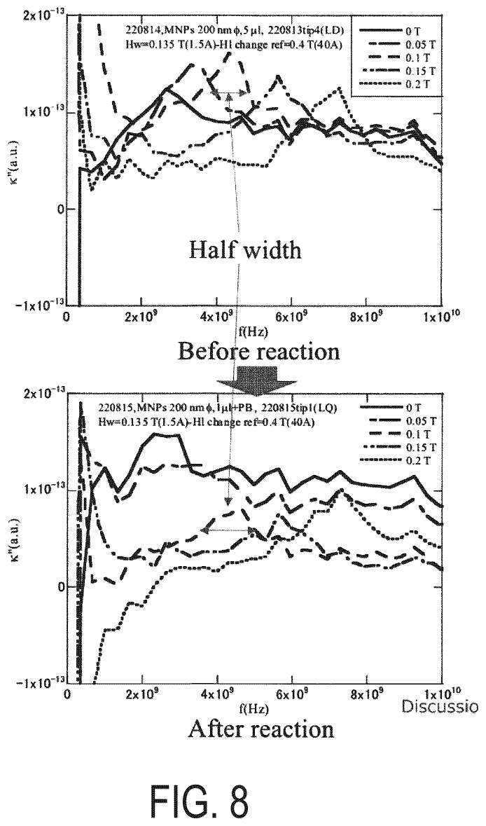

Each diagram in FIG. 8 shows a measurement result example obtained by applying the magnetic susceptibility measurement method according to the present embodiment to the magnetic immunological test. FIG. 8 is graphs of an experiment of a biotin-avidin reaction similar to the antigen-antibody reaction, each showing frequency-dependence of the magnetic susceptibility κ (imaginary part κ″ thereof) of the object to be measured measured by the magnetic susceptibility measurement method of the present embodiment with respect to objects to be measured containing aggregates of the magnetic particles before and after the reaction of biotin, which is an object to be detected that binds to avidin by using the magnetic particles (magnetic nanoparticles of 200 nmf) as avidin-added magnetic markers. FIG. 8(a) shows the magnetic susceptibility before the reaction and FIG. 8(b) shows the magnetic susceptibility after the reaction.

FIG. 8 shows the magnetic susceptibility κ (imaginary part κ″ thereof) when the strength of the magnetic field applied in the magnetization hard axis direction is varied (including a case in which no magnetic field is applied in the magnetization hard axis direction (0.0 T)), and specifically shows the magnetic susceptibility κ when the strength of the magnetic field (magnetic flux density) in the magnetization hard axis direction is 0.0 T (no magnetic field applied), 0.1 T, 0.05 T, 0.1 T, 0.15 T and 0.2 T. In this measurement result example, it can be confirmed that the ferromagnetic resonance (FMR) frequency of the peak value at which the magnetic susceptibility is maximized is shifted in accordance with the strength of the applied magnetic field in the magnetization hard axis direction, and that the peak value is lowered, the sharpness of the peak value is lowered, and the half width is widened between before and after the reaction. As shown in FIG. 8, for example, the half width of the peak value before the reaction is increased twice or more after the reaction at the applied magnetic field 0.1 T, and the object to be detected can be detected by the change in the half width. It is considered that the object to be detected binds to the magnetic particles and the interval between the magnetic particles increases, and thus the uniformity of the magnetic moment decreases. The object to be detected (antigen, biotin) in the antigen-antibody reaction or the biotin-avidin reaction is detected using the measured half width of the peak value of the magnetic susceptibility.

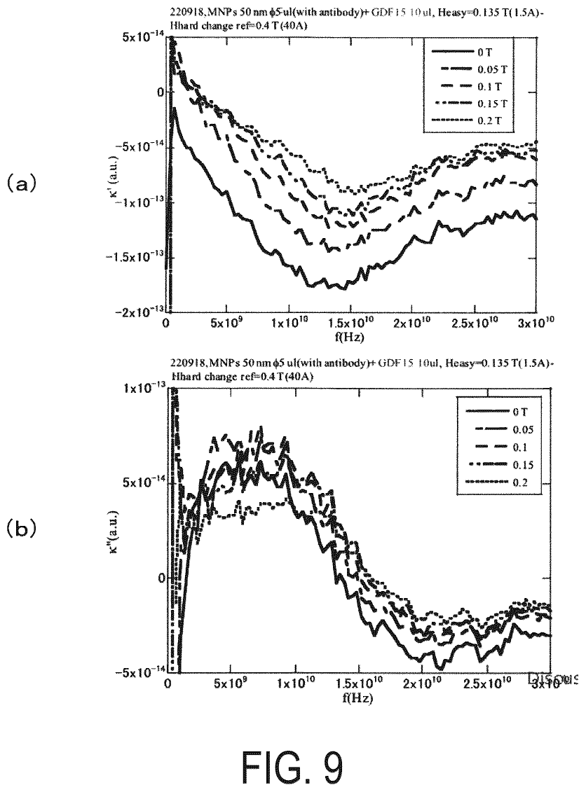

Each diagram in FIG. 9 shows another measurement result example obtained by applying the magnetic susceptibility measurement method according to the present embodiment to a magnetic immunological test. FIG. 9 is graphs of an experiment of detecting the protein GDF15 by the antigen-antibody reaction, each showing frequency-dependence of the magnetic susceptibility κ of the object to be measured measured by the magnetic susceptibility measurement method of the present embodiment with respect to an object to be measured containing aggregates of the magnetic particles reacted with the protein GDF15, which is an object to be detected that binds to the antibody, by using the magnetic particles (magnetic nanoparticles of 50 nmf) as the magnetic markers for the antibody. FIG. 9(a) shows a value of the real part (κ′) of the magnetic susceptibility κ and FIG. 9(b) shows a value of the imaginary part (κ″) of the magnetic susceptibility κ. The protein GDF15 is a protein used for diagnosis of mitochondrial diseases.

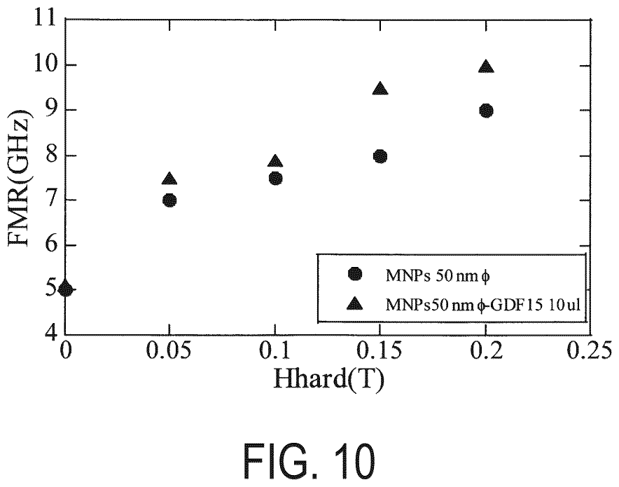

FIG. 10 is a graph showing a relationship between the magnetic susceptibility κ and the ferromagnetic resonance frequencies (FMR) in the magnetic susceptibility measurement shown in FIG. 9, and is a graph showing the ferromagnetic resonance frequency (FMR) at which the magnetic susceptibility κ (imaginary part κ″ thereof) corresponding to the strength of the applied magnetic field H_{hard} in the magnetization hard axis direction becomes the peak value.

FIG. 9 shows the magnetic susceptibility κ when the strength of the magnetic field applied in the magnetization hard axis direction is varied (also including the case in which no magnetic field (0.0 T) is applied in the magnetization hard axis direction), and specifically shows the magnetic susceptibility κ when the strength of the magnetic field (magnetic flux density) in the magnetization hard axis direction is 0.0 T (no magnetic field applied), 0.1 T, 0.05 T, 0.1 T, 0.15 T and 0.2 T. In this measurement result example, although it is difficult to understand from only visual observation of FIG. 9, the ferromagnetic resonance (FMR) frequency of the peak value at which the magnetic susceptibility is maximized is shifted in accordance with the strength of the applied magnetic field in the magnetization easy hard direction. FIG. 10 shows the graph plotting the ferromagnetic resonance frequency corresponding to the strength of the magnetic field.

In FIG. 10, a symbol of black triangle indicates the ferromagnetic resonance frequency of the peak value of the magnetic susceptibility of the object to be measured containing the magnetic particles with the protein GDF15 bound, and a symbol of black circle indicates the ferromagnetic resonance frequency of the peak value of the magnetic susceptibility of the object to be measured containing the magnetic particles with no protein GDF15 bound. In general, it can be confirmed that the ferromagnetic resonance frequency shifts before and after the binding (before and after the reaction) of the protein GDF15 as the applied magnetic field increases. As can be seen from FIG. 10, for example, the shift amount of the ferromagnetic resonance frequency at the applied magnetic fields 0.15 T and 0.2 T is large, and the GDF15 as the object to be detected can be detected by the frequency shift.

In the embodiment of the present invention described above, the method of measuring the magnetic susceptibility of the magnetic particles has been described. However, the magnetic susceptibility has a relationship shown in the following equation (3) with the magnetic permeability of the magnetic particles, and thus the magnetic susceptibility κ can be converted into the magnetic permeability (relative magnetic permeability) μ, and the measuring the magnetic susceptibility can be regarded as the same as measuring the magnetic permeability u.

κ=μ−1 (3)

(μ: relative magnetic permeability)

A signal transmission line included in the probe 10 is not limited to the microstrip line shown in the above-described configuration example, and may be a coplanar line or a coaxial line, for example. The application of the magnetic anisotropy to the magnetic particles is not limited to the case in which the magnetic anisotropy (shape anisotropy) is applied to the magnetic particles in the longitudinal direction thereof by aggregating the magnetic particles in a container such as a microtube elongated in one direction, and for example, magnetic particles each having a flat shape (shape elongated in one direction) in which each particle itself has magnetic anisotropy may be used.

### Reference Example

Hereinafter, a measurement example by another measurement method to which a measurement technique of the present invention can be applied will be described as a reference example to which the present invention can be applied. The measurement example by the measurement method of the reference example is an experiment of detecting the protein GDF15 by the antigen-antibody reaction, in which the magnetic particles (magnetic nanoparticles) are used as the magnetic markers for the antibody, a magnetic field is applied to a sample containing aggregates of the magnetic particles reacted with the protein GDF15, which is the object to be measured (antigen) that bind to antibody, the magnetization is detected by a magnetic field sensor, and the amount (concentration) of the object to be measured (protein GDF15) is measured.

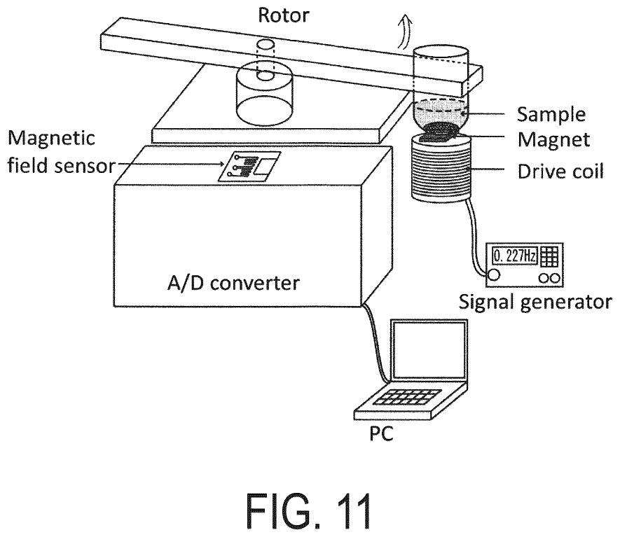

FIG. 11 is a diagram illustrating a schematic configuration of a measurement device for carrying out a measurement example in the reference example. The measurement device includes a rotation mechanism (rotor) for rotating a container which is composed of sample containing the magnetic particles and an object to be measured that can bind to the magnetic particles, a magnetic field generating means for applying, to the sample in the container, a magnetic field (switching magnetic field) whose magnetic field direction is reversed and switched for each revolution in synchronization with a rotation cycle of the container, a magnetic field sensor disposed at a position separated from the magnetic field generating means and detecting a signal corresponding to a magnetic field emitted from the sample contained in the rotatively moving container, and a signal processing means for processing the detection signal.

The magnetic field generating means includes an excitation coil (drive coil) and a signal oscillator (signal generator). Before the container is rotated, a permanent magnet (yoke) is brought in proximity to the bottom of the container to bring the sample into an aggregation state, a magnetic field for switching the magnetic field direction from this state is applied, and the magnetic field from the sample is detected by the magnetic field sensor. For example, the yoke and the excitation coil are disposed concentrically. Preferably, the magnetic field generating means applies the magnetic field such that the strength of the magnetic field increases stepwise for each revolution of the container by the rotation mechanism. The signal processing means includes a signal processing circuit such as an A/D converter and a computer device such as a personal computer (PC). The detailed configuration and operation of the measurement device are disclosed in, for example, JP 2020-159871 A including the inventors of the present application.

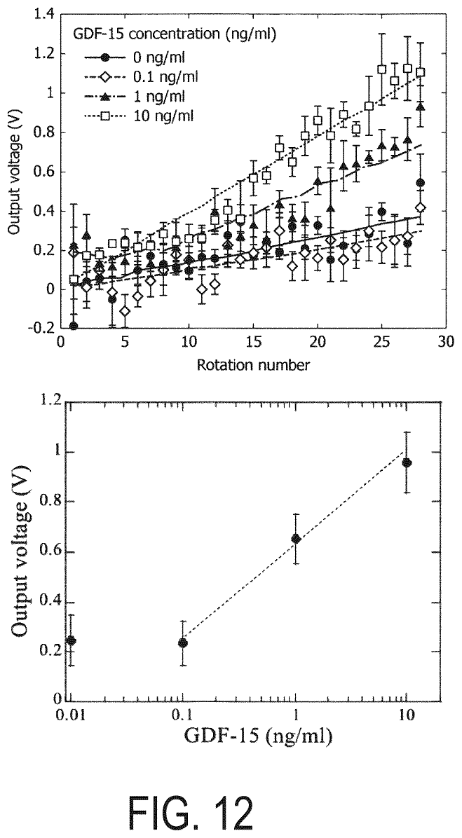

Each diagram in FIG. 12 shows a measurement result example (part 1) of the measurement example in the reference example. The measurement result (part 1) is a measurement result of the measurement experiment in which an antibody obtained by binding magnetic nanoparticles (Nanomag-D, average particle size of 250 nmf) to which Protein A is added and a primary antibody (Anti-GDF15 monoclonal) are produced, and then the antibody and the antigen (protein GDF15) at concentrations 0 ng/ml, 0.1 ng/ml, 1.0 ng/ml, and 10 ng/ml were subjected to the antigen-antibody reaction by using the device configuration illustrated in FIG. 11. FIG. 12(a) shows a relationship between the number of rotations (increase in applied voltage for each rotation) of the container and the output voltage of the magnetic field sensor. FIG. 12(b) shows a relationship between the concentration of the protein GDF15 obtained based on the measurement result of FIG. 12(a) and the output voltage of the magnetic field sensor.

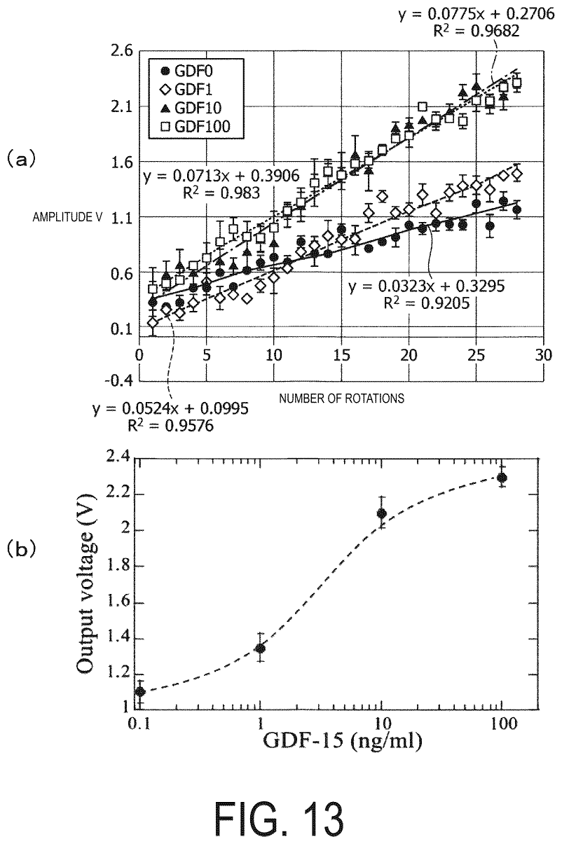

Each diagram in FIG. 13 shows a measurement result (part 2) of the measurement example in the reference example. The measurement result (part 2) is a measurement result of a measurement experiment in which an antibody obtained by binding magnetic nanoparticles added with Protein A (Nanomag-D, average particle size 250 nmf) and a primary antibody (Anti-GDF15 monoclonal) and an antibody obtained by binding magnetic nanoparticles added with Protein A (Nanomag-D, average particle size 50 nmf) and a secondary antibody (Anti-GDF15 polyclonal) were produced (two types of antibodies were produced), and then the two types of antibodies and the antigen (protein GDF15) at concentrations 0 ng/ml, 1.0 ng/ml, 10 ng/ml, and 100 ng/ml were subjected to the antigen-antibody reaction by using the device configuration illustrated in FIG. 11. FIG. 13(a) shows a relationship between the number of rotations (increase in applied voltage for each revolution) of the container and the output voltage of the magnetic field sensor. FIG. 13(b) shows a relationship between the concentration of the protein GDF15 obtained based on the measurement result of FIG. 13(a) and the output voltage of the magnetic field sensor.

In both of the measurement result (part 1) shown in FIG. 12 and the measurement result (part 2) shown in FIG. 13, it has become clear that as the magnetic field applied to amounts shown in FIGS. 12(a) and 13(a) increases (since the applied magnetic field also increases as the number of rotations increases), the output voltage (magnetic signal) of the magnetic field sensor increases, and as the concentration of the proteins GDF15 increases, the degree of increase in the magnetic signal becomes larger. Thus, as shown in FIGS. 12(b) and 13(b), the concentration (amount) of the protein GDF15 can be obtained by the magnitude of the magnetic field to be detected.

When the measurement result (part 1) and the measurement result (part 2) are compared with each other, the error bar in each measurement value in the graph of FIG. 13 is reduced as compared with the graph of FIG. 12, and in the case of the measurement result 2 using two types of antibodies, the magnetic signal is increased as compared with the measurement result 1, and the detection sensitivity and the detection accuracy are improved.

In addition, as shown in FIGS. 12 and 13, it was newly found from this measurement that when the antigen is the protein, the magnetic signal output increases as the antigen concentration increases. This is a physical phenomenon opposite to the phenomenon disclosed in, for example, JP 2020-159871 A, that is, in the phenomenon, the magnetic signal output tends to decrease as the antigen concentration increases when the object to be measured (antigen) in the magnetic immunological test is a bacterium.

It is presumed that the reason why the output tendency of the magnetic signal changes depending on a type of the antigen depends on the size of the antigen. Specifically, in the case of a protein having a size smaller than a size of a bacterium, the size of the protein is relatively small in aggregates of the protein bound to the magnetic nanoparticles, and thus even when the concentration of the protein increases, the magnetic binding between the magnetic nanoparticles is not weakened, and it is presumed that the magnetic signal output also increases, and it is also considered that the effect of promoting the aggregation of the magnetic nanoparticles is generated by the protein. On the other hand, in the case of a bacterium having a size larger than a size of a protein, due to the relatively large size of the bacterium, the distance between the magnetic nanoparticles in aggregates of the bacterium bonded to the magnetic nanoparticles is increased and the magnetic bonding between the magnetic nanoparticles is weakened. Thus, it is presumed that as the concentration of the bacterium increases, the magnetic signal output decreases. According to the measurement example in the above reference example, knowledge can be obtained in which the output tendency of the magnetic signal varies depending on the size of the antigen. The measurement example in the above-described reference example can also be carried out in the same manner by applying the magnetic susceptibility measurement device and the magnetic susceptibility measurement method of the present invention corresponding to FIGS. 1 to 10 to measure the magnetization of the object to be measured.

The present invention is not limited to the above-described embodiments, and it is a matter of course that design changes within a range not departing from the gist including various changes and modifications conceived by a person having ordinary knowledge in the field of the present invention are included in the present invention.

### REFERENCE SIGNS LIST

1: Object to be measured, 1a: Microtube, 3: Cable, 10: Probe, 11: Microstrip conductor, 12: Flexible substrate, 13: Fluorine resin substrate, 14: Ground conductor, 15: Connector, 20: Signal measurement device, 30: Computer device, 40: Magnetization easy axis coil, 50: Magnetization hard axis coil.

1. A magnetic susceptibility measurement device for measuring magnetic susceptibility of an object to be measured containing magnetic particles, the magnetic susceptibility measurement device comprising:
a probe comprising a signal transmission line formed therein, the object to be measured being disposed in proximity to or in contact with the signal transmission line and a magnetization easy axis direction of the object to be measured being orthogonal to the signal transmission line;
a first magnetic field applying unit configured to apply a magnetic field in the magnetization easy axis direction of the object to be measured;
a second magnetic field applying unit configured to apply a magnetic field in a magnetization hard axis direction orthogonal to the magnetization easy axis direction of the object to be measured;
a signal measurement device configured to measure a signal transmitting through the signal transmission line in a state in which magnetic fields are applied by both the first magnetic field applying unit and the second magnetic field applying unit; and
an arithmetic processing means configured to obtain the magnetic susceptibility of the object to be measured based on the signal measured by the signal measurement device.

2. The magnetic susceptibility measurement device according to claim 1, wherein the signal measurement device measures a signal transmitted through the signal transmission line when the magnetic field applied by the first magnetic field applying unit is constant and the strength of the magnetic field applied by the second magnetic field applying unit is changed.

3. The magnetic susceptibility measurement device according to claim 1, wherein the signal transmission line is a microstrip line.

4. A magnetic susceptibility measurement method for measuring magnetic susceptibility of an object to be measured containing magnetic particles, the magnetic susceptibility measurement method comprising:
disposing the object to be measured in proximity to or in contact with a probe comprising a signal transmission line formed therein, the object to be measured being disposed in proximity to or in contact with the signal transmission line, a magnetization easy axis direction of the object to be measured being orthogonal to the signal transmission line;
applying, by a first magnetic field applying means, a magnetic field in the magnetization easy axis direction of the object to be measured;
applying, by a second magnetic field applying means, a magnetic field in a magnetization hard axis direction orthogonal to the magnetization easy axis direction of the object to be measured;
measuring, by a signal measurement device, a signal transmitted through the signal transmission line in a state in which the magnetic fields are applied by both the first magnetic field applying means and the second magnetic field applying means; and
obtaining, by an arithmetic processing means, magnetic susceptibility of the object to be measured based on the signal measured by the signal measurement means.

5. The magnetic susceptibility measurement device according to claim 4, wherein, in the measuring the signal, a signal transmitted through the signal transmission line when the magnetic field applied by the first magnetic field applying unit is constant and the strength of the magnetic field applied by the second magnetic field applying unit is changed is measured.

6. The magnetic susceptibility measurement method according to claim 4, wherein, in the obtaining the magnetic susceptibility, the magnetic susceptibility corresponding to a predetermined frequency band is obtained, and an object to be detected bound to the magnetic particles contained in the object to be measured is detected using a peak value of the magnetic susceptibility in the predetermined frequency band.

7. The magnetic susceptibility measurement method according to claim 4, wherein, in the obtaining the magnetic susceptibility, the magnetic susceptibility corresponding to a predetermined frequency band is obtained, and an object to be detected bound to the magnetic particles contained in the object to be measured is further detected based on a frequency at which the magnetic susceptibility becomes peak.

8. A magnetic susceptibility control method for controlling magnetic susceptibility of an object to be measured containing magnetic particles, the magnetic susceptibility control method comprising:
magnetizing, by a first magnetic field applying means, the object to be measured in an easy axis direction of the object to be measured;
applying, by a second magnetic field applying means, a magnetic field in a magnetization hard axis direction orthogonal to the magnetization easy axis direction of the object to be measured; and
varying, by a control means, the strength of the magnetic field applied by the second magnetic field applying means to control to change the magnetic susceptibility of the object to be measured.

9. The magnetic susceptibility control method according to claim 8, wherein the object to be measured is a protein, and the size of the protein is estimated based on an output tendency of the magnetic susceptibility.
